(19) United States
(12) Patent Application Publication
(10) Pub. No.: US 2004/0048849 A1
(43) Pub. Date: Mar. 11, 2004

(54) Cyclin-dependent kinase (cdk) and glycolene synthase kinase-3 (gsk-3) inhibitors

(76) Inventors: Gregoire Prevost, Antony (FR); Marie-Odile Lonchampt, Chevilly-Larue (FR); Sun Kim, Needham, MA (US); Barru Morgan, Franklin, MA (US); Gerard Ulibarri, Ottawa (CA); Christophe Thurieau, Paris (FR)

Correspondence Address: MUSERLIAN AND LUCAS AND MERCANTI, LLP, 475 PARK AVENUE SOUTH, NEW YORK, NY, 10016, US

(21) Appl. No.: 10/433,857

(22) PCT Filed: Dec. 19, 2001
(86) PCT No.: PCT/FR01/04048

(30) Foreign Application Priority Data
Dec. 20, 2000 (FR) 00/16632
Oct. 23, 2001 (FR) 01/13636

(51) Int. Cl. A61K 31/55; A61K 31/53

(52) U.S. Cl. 514/217.05; 514/246

(57) ABSTRACT

The invention concerns novel cyclin-dependent kinase (CDK) and glycolene synthase kinase-3 (GSK-3) inhibitors, corresponding to general formula (I), wherein: A represents a hydrogen atom, a halogen atom, a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or a -L-NR¹R² radical wherein L represents an alkylene radical and R¹ and R² are selected independently among a hydrogen atom and an alkyl radical or R¹ and R² together with the nitrogen bearing them form a heterocycle with 5 to 7 members optionally substituted; X represents a hydrogen atom, an alkylthio, aralkylthio, alkylthioxo or aralkylthioxo radical, or a NR⁴R⁵ radical wherein R⁴ represents an alkyl radical, a hydroxyalkyl radical, a cycloalkyl radical optionally substituted, an aralkyl radical whereof the aryl radical is optionally substituted, or R⁴ represents a heteroaryl or heteroarylalkyl radical, the heteroaryl radical of the heteroaryl or heteroarylalkyl radicals being optionally substituted; Y represents NH or an oxygen atom; Z represents a bond or an alkyl or alkylthioalkyl radical; and Ar represents a carbocyclic aryl radical optionally substituted, a heterocyclic aryl radical optionally substituted or a piridiniumolate radical; or are pharmaceutically acceptable salts of said compounds.

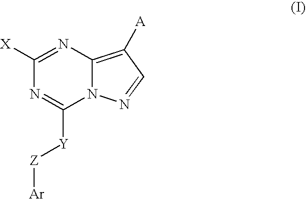

A subject of the present invention is new inhibitors of cyclin-dependent kinases, and in particular cyclin B1/cdc2 and glycogen synthase kinase-3 (GSK-3).

Control of the transition between the different phases of the cell cycle of mitosis or meiosis is provided by a set of proteins the enzymatic activities of which are associated with different states of phosphorylation. These states are controlled by two large classes of enzymes: the kinases and the phosphatases.

Synchronization of the different phases of the cell cycle thus allows reorganisation of the cell architecture at each cycle in the whole of the living world (microorganisms, yeasts, higher organisms, plants). Among the kinases, the cyclin-dependent kinases (CDKs) play a major role in this control of the cell cycle. The CDKs are complexes in which the kinases support the enzymatic activity and the associated cyclins have a regulatory action on the latter. The association between these proteins is not permanent, these associations vary during the cycle in precise time windows. Several cyclins and CDKs coexist in the cell, but the associations between the cyclins and the CDKs are specific. To date, at least ten CDKs have been described (CDK1-X) (Detivaud et al., Eur. J Biochem. (1999), 264, 55-66). The enzyme cdc2 is also called CDK1 (Meijer et al., Eur. J Biochem. (1997), 243, 527-536). With the exception of CDK3, each of the CDKs is specifically associated with one or more members of the family of cyclins:

cyclin A: CDK1 and CDK2;

cyclins B1-B3: CDK1;

cyclin C: CDK8;

cyclins D1-D3: CDK1, CDK2, CDK4, CDK5 and CDK6;

cyclin E: CDK2;

cyclin H: CDK7;

cyclin T: CDK9.

The CDK partners of the cyclins F, G and I have not yet been identified. Other kinases close to cdc2 and other cyclins have been identified and the characterization of their functions is in progress such as for example in parasites (Le Roch et al., J Biol. Chem. (2000), 275, 8952-8958) or also in the herpes viruses (Card et al., EMBO (2000), 19, 2877-2888).

Moreover, the enzymatic activity of these different CDKs is controlled by two other families of enzymes which work in opposition (Jessus and Ozon, Prog. Cell Cycle Res. (1995), 1, 215-228). The first includes kinases such as Wee1 and Mik1 which deactivate CDKs by phosphorylating certain amino acids (Den Haese et al., Mol. Biol. Cell (1995), 6, 371-385). The second includes phosphatases such as the family of Cdc25s which activates certain CDKs by dephosphorylating tyrosine and threonine residues of CDKs (Gould et al., Science (1990), 250, 1573-1576). It should be noted that cell entry can be produced without activation of the kinase cdc2 in cells treated with okadaic acid suggesting that the phosphatase cdc25C and other kinases may play a role in this process (Gowdy et al., J Cell Sci. (1998), 111, 3401-3410).

In order to complete this control of the cell cycle, different endogenous inhibitors of CDKs have been identified: p16ink4A, p15ink4B, p18ink4C, p27kip1, p57kip2, p21cip1 (Linares-Cruz et al., Proc. Natl. Acad. Sci. U.S.A. (1998), 95, 1131-1135; Goubin and Ducommun, Oncogene (1995), 10, 2281-2287). The expression of these endogenous inhibitors is very often altered in tumorous cells.

Many compounds blocking the kinase activity of CDKs are known (Meijer and Kim, Methods Enzymol. (1997), 283, 113-128). They have been studied in several therapeutic fields such as oncology to prevent the division of tumorous cells (McDonald and el-Deiry, Int. J Oncol. (2000), 16, 871-886), neurobiology to prevent the natural or chemically-induced apoptosis of normal cells (for example neurones) (Maas et al., J Neurochem. (1998), 70, 1401-1410; Park et al., J Neurosci. (1997), 17, 1256-1270), nephrology to restore the renal function which has been altered in case of glomerulonephritis (Pippin et al., J Clin. Invest. (1997), 100, 2512-2520) and parasitology to block the reproduction cycle of parasites (Knockaert et al., Chem. Biol. (2000), 7, 411-422; Le Roch et al., J Biol. Chem. (2000), 275, 8952-8958).

The inhibitors of cyclin-dependant kinases are therefore capable of being used as medicaments, in particular in the treatment of the diseases/disorders described in Meijer et al., Pharmacol. Ther. (1999), 82, 279-284, and in particular:

to inhibit tumorous proliferation when used alone or in combination with other treatments;

to inhibit the proliferation of normal cells when used alone or in combination with other treatments (for example: atherosclerosis, angiogenesis, psoriasis or restenosis);

in the prevention of spontaneous alopecia;

in the prevention of alopecia induced by exogenous products;

in the prevention of radiation-induced alopecia;

in the prevention of spontaneous or induced apoptosis of normal cells (ischemia);

in the prevention of meiosis and fecundation;

in the prevention of maturation of oocytes;

in the treatment of viral or retroviral infections (herpes, AIDS, cytomegalovirus);

in the prevention and treatment of neurodegenerative diseases (for example tauopathies and in particular Alzheimer's disease);

in the prevention and treatment of parasites (proliferation of protozoa, for example Trypanosoma, Toxoplasma or Plasmodium);

in the treatment of myopathies;

and more generally in the treatment of all diseases/all disorders corresponding to the reported uses for inhibitors of CDKs.

As regards the glycogen synthase kinase-3 (GSK-3) enzyme (Parker et al., Eur. J Biochem. 1983), 130, 227-234) it is a serine/threonine kinase enzyme. There are two isoforms α and β originating from two separate genes. Isoform α codes for a polypeptide of 51 kd. Isoform β codes for a polypeptide of 47 kd having an 85% amino acids homology with GSK-3 α (Woodgett, EMBO (1990), 9, 2431-2438.

The expression levels of messengers for the α and β isoforms of GSK-3 are predominant in the testicles, the thymus, the prostate and the ovaries but low in the lung and the kidney. Analysis of the detection of proteins in the various tissues shows a lack of correlation between the transcription and the translation (Lau et al., J Pept. Res. (1999), 54, 85-91).

GSK-3 is in an activated form in the cells where it inhibits glycogen synthase by direct phosphorylation (Eldar-Finkelman et al., Proc. Natl. Acad. Sci. U.S.A. (1996), 93, 10228-10233)(3). Insulin inhibits GSK-3 and leads to the activation of glycogen synthase. The inhibition of GSK-3 can be observed with other growth factors such as Insulin-like Growth Factor-I (IGF-I) or Epidermal Growth Factor (EGF).

Moreover, GSK-3 participates in other biological processes including control of the cell cycle (Diehl et al., Genes & Dev. (1998), 12, 3499-3511), cellular distribution of β-catenin (Yost et al., Genes & Dev. (1996), 10, 1443-1454), cell survival and activation of Nf-kappaB in the control of apoptosis (Hoeflich et al., Nature (2000), 406, 86-90), metabolism of glucose (Summers et al., J Biochem. (1999), 274. 17934-17940), phosphorylation of the tau protein (Spittaels et al., J Biol. Chem., Sep. 27, 2000), or also the dynamics of microtubules (Krylova et al., J Cell Biol. (2000), 151(1), 83-94).

The role of GSK-3 is still being studied and there are probably numerous interventions that have yet to be reported.

Among the molecules currently reported as inhibitors of GSK-3, principally there can be mentioned:

lithium, a therapeutic agent used in the treatment of depression for very many years, which is a direct inhibitor of GSK-3; in addition to these effects on depression, lithium can modulate the proliferation of normal or tumorous cells (Cui et al., Brain Res. Dev. Brain Res. (1998), 111(2), 177-88);

the compounds SB-216763 and SB-415286 specifically inhibit GSK-3α and GSK-3α in vitro with Kᵢs of the order of nM. They stimulate the synthesis of glycogen in human liver cells by inhibiting the GSK-3 cellular activity measured by activation of glycogen synthase which is the direct target of GSK-3 (Coghlan et al., Chem. Biol. (2000), 7(10), 793-803).

the majority of the inhibitors of CDK, with the exception of those derived from purines, are reported as powerful inhibitors of GSK-3 (Meijer, Supplement to Cancer Clinical Research (November 2000), 6, Proceedings of the NCI-EORTC-ACCR Symposium, 043).

There are also inhibitors of CDKs which are not inhibitors of GSK-3, such as derivatives of purines (roscovitine, olomucine, purvalanol etc.) and butyrolactone. The specific inhibition values of various products on the two classes of enzymes are reported in Leclerc et al., J Biol. Chem., September 2000.

There are numerous potential therapeutic applications of inhibitors of GSK-3 (Ferkey et al., Dev. Biol. (2000), 225(2), 471-479):

depression;

humoral disorders (Manji et al., J Clin. Psychiatry (2000), 61(Suppl. 9), 82-96);

the neurodegenerative disorders such as Parkinson's disease;

tauopathies, pathologies where the tau protein is hyperphosphorylated such as in Alzheimer's disease or certain dementias;

proliferative diseases, and in particular cancer; and

diabetes (Nikoulina et al., Diabetes (2000), 49(2), 263-71).

Certain derivatives of triazolopyrazines with simpler structures have already been used in therapy, for example both as inhibitors of phosphodiesterases (Patents U.S. Pat. No. 3,846,423 and U.S. Pat. No. 3,865,824), and as antagonists of the corticotropin-releasing factor (CRF) (Patent Applications PCT WO 98/08847 and WO 99/67247) or also in the treatment of respiratory disorders (Patent U.S. Pat. No. 3,995,039), gastro-intestinal disorders (Patent U.S. Pat. No. 4,565,815) or cardio-vascular and circulatory disorders (Patent U.S. Pat. No. 5,356,894).

The compounds corresponding to general formula (I)

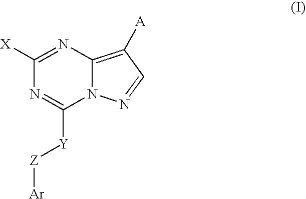

in racemic, enantiomeric form or any combination of these forms, in which

A represents a hydrogen atom, a halogen atom, a formyl, cyano, guanidinoaminomethylenyl, (1,3-dihydro-2-oxoindol)-3-ylidenemethyl, alkylcarbonyl, aralkylcarbonyl or heteroaralkylcarbonyl radical, or also an -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, complementary members being chosen independently from the group comprising —CH₂—, —NR³—, —S— and —O—, R³ representing independently each time that it occurs a hydrogen atom or an alkyl radical;

X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by a radical or radicals chosen from the alkyl, hydroxy and amino radicals, an aralkyl radical the aryl radical of which is optionally substituted by a radical or radicals chosen from a halogen atom, the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical;

Y represents NH or an oxygen atom;

Z represents a bond or an alkyl or alkylthioalkyl radical; and

Ar represents a carbocyclic aryl radical optionally substituted 1 to 3 times by radicals chosen independently from a halogen atom, the cyano radical, the nitro radical, an alkyl or alkoxy radical and an NR⁷R⁸ radical in which R⁷ and R⁸ represent independently a hydrogen atom or an alkyl radical or R⁷ and R⁸ taken together with the nitrogen atom which carries them form a heterocycle with 5 to 7 members, the complementary members being chosen independently from the group comprising —CH₂—, —NR⁹—, —S—and —O—, R⁹ representing independently each time that it occurs a hydrogen atom or an alkyl radical,

or also Ar represents a heterocyclic aryl radical containing 5 or 6 members and the heteroatom or heteroatoms of which are chosen from nitrogen, oxygen or sulphur atoms, said heteroatoms being able to be optionally oxidized (Ar can represent for example the oxidopyridyl radical) and said heterocyclic aryl radical being able to be optionally substituted by a radical or radicals chosen independently from the alkyl, aminoalkyl, alkylaminoalkyl and dialkylaminoalkyl radicals;

or the pharmaceutically acceptable salts of the compounds of general formula (I) can be used for preparing a medicament intended to inhibit the cyclin-dependent kinases (CDK).

According to a preferred variant of the invention, the compounds of general formula (I) can be used for preparing a medicament intended to inhibit both cyclin-dependent kinases (CDK) and glycogen synthase kinase-3 (GSK-3).

By alkyl, when it is not specified otherwise, is meant a linear or branched alkyl radical containing 1 to 6 carbon atoms. By cycloalkyl, when it is not specified otherwise, is meant a cycloalkyl radical containing 3 to 7 carbon atoms. By carbocyclic or heterocyclic aryl, when it is not specified otherwise, is meant a carbocyclic or heterocyclic system containing from one to three condensed rings at least one of which is an aromatic ring, a system being called heterocyclic when at least one of the rings which comprises it contains a heteroatom or heteroatoms (O, N or S). By aryl, when it is not specified otherwise, is meant a carbocyclic aryl radical. By heteroaryl is meant a heterocyclic aryl radical

By alkylcarbonyl, aralkylcarbonyl, heteroaralkylcarbonyl, alkylthio, aralkylthio, alkylthioxo, aralkylthioxo, hydroxyalkyl, alkylthioalkyl, aralkyl, heteroaralkyl, aminoalkyl, alkylaminoalkyl and dialkylaminoalkyl radicals is meant respectively the alkylcarbonyl, aralkylcarbonyl, heteroaralkylcarbonyl, alkylthio, aralkylthio, alkylthioxo, aralkylthioxo, hydroxyalkyl, alkylthioalkyl, aralkyl, heteroaralkyl, aminoalkyl, alkylaminoalkyl and dialkylaminoalkyl radicals the aryl, heteroaryl and alkyl radicals of which have the meanings indicated previously.

By linear or branched alkyl having 1 to 6 carbon atoms, is meant in particular the methyl, ethyl, propyl, isopropyl, butyl, isobutyl, sec-butyl and tert-butyl, pentyl, neopentyl, isopentyl, hexyl, isohexyl radicals. By cycloalkyl, is meant in particular the cyclopropyl and cyclohexyl radicals. By carbocyclic aryl, is meant in particular the phenyl and naphthyl radicals. By heterocyclic aryl, is meant in particular the pyrrolyl, furannyl, thienyl, pyridyl, imidazolyl, oxazolyl, thiazolyl, indolyl and quinolyl radicals. Finally, by halogen, is meant the fluorine, chlorine, bromine or iodine atoms.

By pharmaceutically acceptable salt is meant in particular the addition salts with inorganic acids such as hydrochloride, hydrobromide, hydroiodide, sulphate, phosphate, diphosphate and nitrate or with organic acids such as acetate, maleate, fumarate, tartrate, succinate, citrate, lactate, methanesulphonate, p-toluenesulphonate, pamoate and stearate. Also included in the scope of the present invention, when they can be used, are the salts formed from bases such as sodium or potassium hydroxide. For other examples of pharmaceutically acceptable salts, reference can be made to “Salt selection for basic drugs”, Int. J Pharm. (1986), 33, 201-217.

In certain cases, the compounds according to the present invention can contain asymmetrical carbon atoms. As a result, the compounds according to the present invention have two possible enantiomeric forms, i.e. the “R” and “S” configurations. The present invention includes the two enantiomeric forms and all combinations of these forms, including the racemic “RS” mixtures. In an effort to simplify matters, when no specific configuration is indicated in the structural formulae, it should be understood that the two enantiomeric forms and their mixtures are represented.

In particular, the compounds of general formula (I) defined previously, or their pharmaceutically acceptable salts, can be used for preparing a medicament intended to treat the following natural diseases/disorders/phenomena: tumorous proliferation, the proliferation of normal cells, spontaneous alopecia, alopecia induced by exogenous products, radiation-induced alopecia, the spontaneous or induced apoptosis of normal cells (ischemia), meiosis, fertilization, maturation of oocytes, viral or retroviral infections (herpes, AIDS, cytomegalovirus), neurodegenerative diseases (for example tauopathies such as Alzheimer's disease), the proliferation of parasites (proliferation of protozoa, for example Trypanosoma, Toxoplasma or Plasmodium) and myopathies. More particularly, the compounds of general formula (I) defined previously, or their pharmaceutically acceptable salts, can be used for preparing a medicament intended to treat the following natural diseases/disorders/phenomena: tumorous proliferation, the proliferation of normal cells, in particular the restenosis, and tauopathies such as Alzheimer's disease.

Preferably, the compounds according to the invention are such that they have at least one of the following characteristics:

A represents a hydrogen atom, a halogen atom, a formyl, cyano, nitro, guanidinoaminomethylenyl, (1,3-dihydro-2-oxoindol)-3-ylidenemethyl, alkylcarbonyl or aralkylcarbonyl radical, or also an -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, the complementary members being chosen independently from the group comprising——H₂—, —NR³—, —S— and —O—, R³ representing independently each time that it occurs a hydrogen atom or an alkyl radical;

X represents a hydrogen atom, an alkylthio or alkylthioxo radical, or also an NR⁴R⁵ radical in which R⁴ represents an alkyl radical, a hydroxyalkyl radical, a cycloalkyl radical optionally substituted by an amino radical or radicals, an aralkyl radical the aryl radical of which is optionally substituted by a radical or radicals chosen from a halogen atom and the alkyl or alkoxy radical, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or then R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical.

More preferentially, the compounds according to the invention are such that they have at least one of the following characteristics:

A represents a atom halogen, a formyl, guanidinoaminomethylenyl, (1,3-dihydro-2-oxoindol)-3-ylidenemethyl or alkylcarbonyl radical, or also an -L-NR¹R² radical in which L represents a methylene radical and R¹ and R² are chosen independently from a hydrogen atom and an alkyl radical or R¹ and R² taken together with the nitrogen atom which carries them form a heterocycle with 5 to 7 members, the complementary members being chosen independently from the group comprising —CH₂—, —NR³— and —O—, R³ representing independently each time that it occurs a hydrogen atom or an alkyl radical;

X represents an alkylthio or alkylthioxo radical, or also an NR⁴R⁵ radical in which R⁴ represents an alkyl radical, a hydroxyalkyl radical, a cycloalkyl radical optionally substituted by an amino radical or radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶— and —O—, R⁶ representing independently each time that it occurs a hydrogen atom or an alkyl or hydroxyalkyl radical;

Z represents a bond or an alkyl radical;

Ar represents a carbocyclic aryl radical optionally substituted 1 to 3 times by the radicals chosen independently from a halogen atom and an NR⁷R⁸ radical in which R⁷ and R⁸ represent independently a hydrogen atom or an alkyl radical or R⁷ and R⁸ taken together with the nitrogen atom which carried them form a heterocycle with 5 to 7 members, the complementary members being chosen independently from the group comprising —CH₂—, —NR⁹— and —O—, R⁹ representing independently each time that it occurs a hydrogen atom or an alkyl radical,

or also Ar represents a heterocyclic aryl radical containing 5 or 6 members and the heteroatom or heteroatoms of which are chosen from nitrogen and oxygen atoms, said heteroatoms being able to be optionally oxidized and said heterocyclic aryl radical being able to be optionally substituted by a radical or radicals chosen independently from the alkyl, aminoalkyl, alkylaminoalkyl and dialkylaminoalkyl radicals.

Also more preferentially, the compounds according to the invention are such that they have at least one of the following characteristics:

A represents a halogen atom, a formyl, guanidinoaminomethylenyl, (1,3-dihydro-2-oxoindol)-3-ylidenemethyl or alkylcarbonyl radical, or also an -L-NR¹R² radical in which L represents a methylene radical and R¹ and R² are chosen independently from a hydrogen atom and an alkyl radical or R¹ and R² taken together with the nitrogen atom which carries them form a heterocycle with 5 to 7 members, the complementary members being chosen independently from the group comprising —CH₂—, —NR³— and —O—, R³ representing independently each time that it occurs a hydrogen atom or an alkyl radical;

X represents an alkylthio (and preferably methylthio) or alkylthioxo (and preferably methylthioxo) radical, or also an NR⁴R⁵ radical in which R⁴ represents an alkyl radical, a hydroxyalkyl radical, a cycloalkyl (and preferably cyclohexyl) radical optionally substituted by an amino radical or radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂— and —NR⁶—, R⁶ representing independently each time that it occurs a hydrogen atom or an alkyl or hydroxyalkyl radical;

Y represents NH;

Z represents a bond or a —CH₂— radical;

Ar represents a carbocyclic aryl radical (said carbocyclic aryl preferably being a phenyl radical) optionally substituted 1 to 3 times by the radicals chosen independently from a halogen atom and an NR⁷R⁸ radical in which R⁷ and R⁸ represent independently a hydrogen atom or an alkyl radical or R⁷ and R⁸ taken together with the nitrogen atom which carries them form a heterocycle with 5 to 7 members, the complementary members being chosen independently from the group comprising —CH₂— and —NR⁹—, R⁹ representing independently each time that it occurs an alkyl radical,

or also Ar represents a heterocyclic aryl radical containing 5 or 6 members and the heteroatom or heteroatoms of which are chosen from nitrogen and oxygen atoms, (said heterocyclic aryl preferably being a pyridyl radical), said heteroatoms being able to be optionally oxidized and said heterocyclic aryl radical being able to be substituted by a radical or radicals chosen independently from the alkyl, aminoalkyl, alkylaminoalkyl and dialkylaminoalkyl radicals.

The compounds described in Examples 1 to 33 hereafter are moreover particularly preferred. Also more particularly preferred are the compounds of Examples 1 to 8, 11, 12, 15, 1826 and 33

Moreover a subject of the invention is, as medicaments, the compounds of general formula (II)

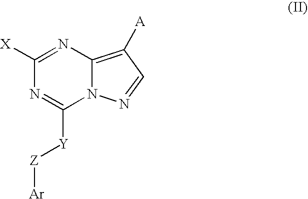

in racemic, enantiomeric form or any combination of these forms, in which

A represents a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or also an -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, the complementary members being chosen independently from the group comprising —CH₂—, —NR³—, —S— and —O—, R³ representing independently each time that it occurs a hydrogen atom or an alkyl radical;

X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by a radical or radicals chosen from the alkyl, hydroxy and amino radicals, an aralkyl radical the aryl radical of which is optionally substituted by a radical or radicals chosen from a halogen atom the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical;

Y represents NH or an oxygen atom;

Z represents a bond or an alkyl or alkylthioalkyl radical; and

Ar represents a carbocyclic aryl radical optionally substituted 1 to 3 times by the radicals chosen independently from a halogen atom, the cyano radical, the nitro radical, an alkyl or alkoxy radical and an NR⁷R⁸ radical in which R⁷ and R⁸ represent independently a hydrogen atom or an alkyl radical or R⁷ and R⁸ taken together with the nitrogen atom which carries them form a heterocycle with 5 to 7 members, the complementary members being chosen independently from the group comprising —CH₂—, —NR⁹—, —S— and —O—, R⁹ representing independently each time that it occurs a hydrogen atom or an alkyl radical,

or also Ar represents a heterocyclic aryl radical comprising 5 or 6 members and the heteroatom or heteroatoms of which are chosen from nitrogen, oxygen or sulphur atoms, said heteroatoms being able to be optionally oxidized (Ar can represent for example the oxidopyridyl radical) and said heterocyclic aryl radical being able to be optionally substituted by a radical or radicals chosen independently from the alkyl, aminoalkyl, alkylaminoalkyl and dialkylaminoalkyl radicals;

it being understood, however, that when A does not represent a cyano, nitro or guanidinoaminomethylenyl radical then:

either Z represents an alkyl or thioalkyl radical;

or X represents an NR⁴R⁵ radical in which R⁴ represents an aralkylthio, aralkylthioxo or hydroxyalkyl radical, one of the alkyl, alkylthio or alkylthioxo radicals containing 2 to 5 carbon atoms, a cycloalkyl radical optionally substituted by a radical or radicals chosen from the alkyl, hydroxy and amino radicals, an aralkyl radical the aryl radical of which is optionally substituted by a radical or radicals chosen from a halogen atom and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5-to 7 members, the complementary members being chosen independently from the group comprising —CH₂—, —NR⁶—, —S— and —O—, R⁶ representing independently each time that it occurs a hydrogen atom or an alkyl or hydroxyalkyl radical;

or the pharmaceutically acceptable salts of the compounds of general formula (II).

In particular, the invention relates as medicaments to the compounds of Examples 3 to 33.

It also relates to the pharmaceutical compositions containing, as active ingredient, at least one compound of general formula (II), one of the compounds of Examples 3 to 33, or a pharmaceutically acceptable salt of one of the latter. It finally relates to, as new industrial products, the compounds of general formula (II) or their salts or one of the compounds of Examples 3 to 33 or a salt of one of the latter.

Generally, the same preferences as those indicated previously for the uses of the compounds of general formula (I) are applicable mutatis mutandis to the compounds of general formula (II) of medicaments, pharmaceutical compositions and products according to the invention.

The pharmaceutical compositions containing a compound of the invention can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups. Appropriate liquid supports can be, for example, water, organic solvents such as glycerol or glycols, similarly their mixtures, in varying proportions, in water.

The administration of a medicament according to the invention can be done by topical, oral, parenteral route, by intramuscular injection, etc.

The administration dose envisaged for a medicament according to the invention is comprised between 0.1 mg to 10 g according to the type of active compound used.

In accordance with the invention, the compounds of general formula (I) can be prepared by the processes described below.

Preparation of the Compounds of General Formula (I):

A certain number of triazolopyrazines of general formula (I) can be easily prepared according to the procedures described in the Patent U.S. Pat. No. 4,565,815.

The other compounds of general formula (I) according to the invention can be prepared in a few stages, Diagram 1, starting from the compounds of general formula (III) in which A′ represents a hydrogen atom or a halogen atom and X′ represents a hydrogen atom or an alkylthio radical. The preparation of the compounds of general formula (III) is described in the Patent U.S. Pat. No. 4,565,815 or in Kobe et al., J Het. Chem. (1974), 11(2), 199 and subsequent.

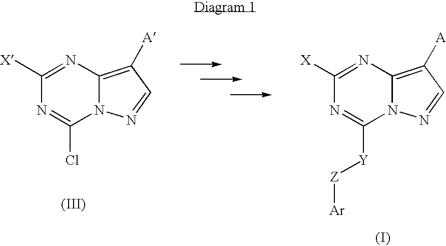

Different cases must be considered depending on the nature of the substituents A, X and Y-Z-Ar of the compounds of general formula (I).

Preparation of the Compounds of General Formula (I) in which A Represents a Hydrogen Atom or a Halogen Atom:

Preparation of the Compounds of General Formula (I) in which X Represents a Hydrogen Atom or Alkylthio:

In this case, the starting compound of general formula (III) is such that X represents H or alkylthio and A represents H or a halogen atom Hal. The synthesis strategy is summarized in Diagram 2 below.

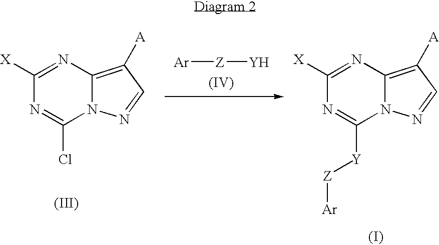

The compound of general formula (III) is subjected to a nucleophilic substitution reaction with the compound of general formula (IV) in order to produce the compound of general formula (I). The reaction can, if necessary, be carried out in a solvent such as chloroform.

Preparation of the Compounds of General Formula (I) in which X Represents an NR⁴R⁵ Radical:

In this case, the starting compound of general formula (III) is such that X′ represents alkylthio and preferably methylthio. The synthesis strategy is summarized in Diagram 3 below.

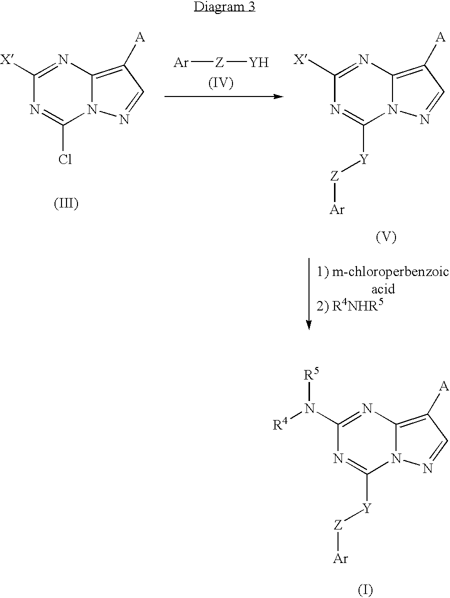

The compound of general formula (III) is firstly subjected to a substitution reaction with the alcohol or the amine of general formula (IV) in order to produce the compound of general formula (V). The compound of general formula (V) is then treated with meta-chloroperbenzoic acid then with the amine of general formula R⁴NHR⁵ in order to finally produce the compound of general formula (I). These reactions are preferably carried out in a solvent such as chloroform.

Preparation of the Compounds of General Formula (I) in which X Represents an Alkylthioxo Radical:

This preparation is carried out in a similar fashion to that described in Diagram 3, the only difference being that the thioxo derivative is isolated during the second stage instead of being substituted by the amine of general formula R⁴NHR⁵ (cf. Diagram 3a).

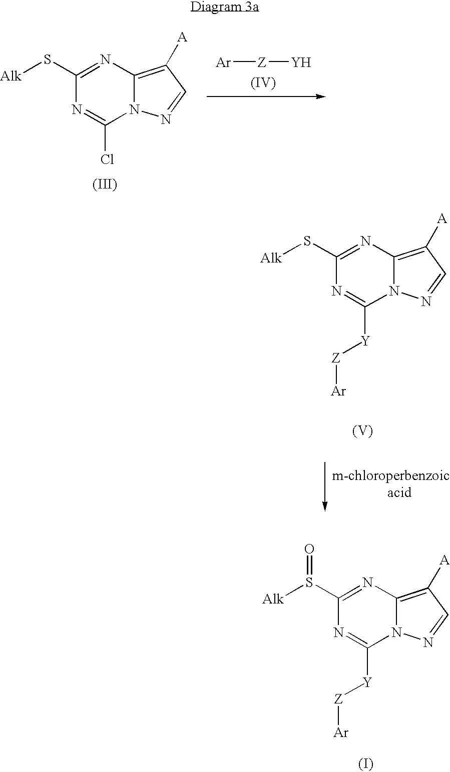

Preparation of the Compounds of General Formula (I) in which A does not Represent a Hydrogen Atom or a Halogen Atom:

Preparation of the Compounds of General Formula (I) in which A Represents a —CH₂—NR¹R² Radical:

When A represents an -L-NR¹R² radical in which L represents —CH₂—, the compound of general formula (VI) represented in Diagram 4 is used for example as starting compound. This compound is a compound of general formula (I) in which A represents H and its synthesis has therefore been described previously. The compound of general formula (VI) is for example firstly treated with an excess of (chloromethylene)-dimethylammonium chloride in an aprotic polar solvent such as an acetonitrile-dimethylformamide mixture. This allows the compounds of general formula (I) to be obtained in which A represents the formyl radical. These compounds allow a person skilled in the art to construct different compounds of general formula (I) with varied A radicals using standard chemical reactions.

In the particular case where A represents an -L-NR¹R² radical in which L represents —CH₂— and R¹ and R² are methyl groups, the compound of general formula (I) can be directly obtained from the compound of general formula (VI) by reaction with (chloromethylene)-dimethylammonium chloride in excess followed by the action of NaBH₄.

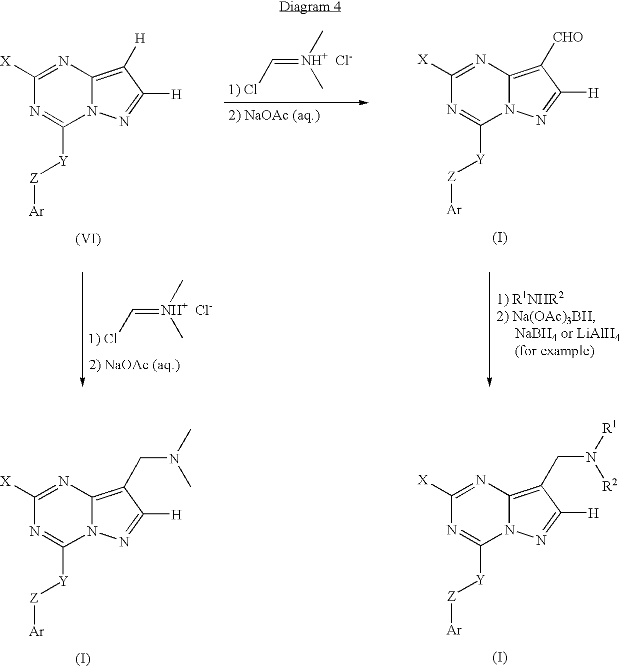

Preparation of the Compounds of General Formula (I) in which A Represents an -L-NR¹R² Radical:

These compounds can be prepared in a standard fashion from the compound of general formula (VI), for example according to the process represented in Diagram 5. The compound of general formula (VI) can for example be treated at low temperature (for example at −78° C.) successively with butyllithium in an aprotic polar solvent such as ethyl ether or tetrahydrofuran then the compound of general formula (VII) in which Hal represents a halogen atom, before being hydrolyzed with slightly acidified water in order to produce the compound of general formula (I) in which A represents an -L-NR¹R² radical.

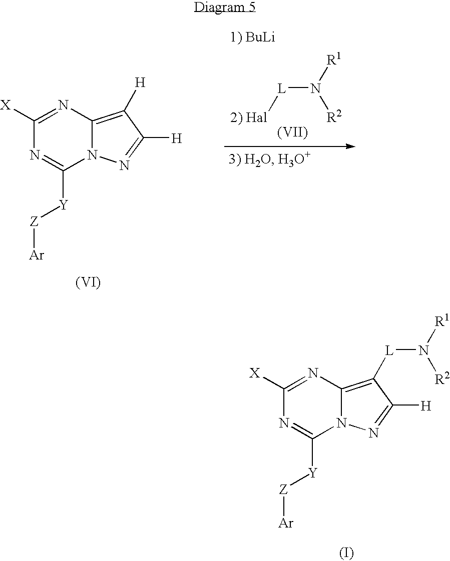

Preparation of the Compounds of General Formula (I) in which A Represents an Alkylcarbonyl, Aralkylcarbonyl, Heteroaralkylcarbonyl Radical:

When one wishes to obtain a compound of general formula (I) in which A is a 'CO—Δ radical in which Δ represents an alkyl, aralkyl or heteroaralkyl radical, the compound of general formula (VI) is treated, Diagram 6, with the compound of general formula Δ-COCl in the presence of ALCl₃ in a suitable solvent, for example in dichloromethane.

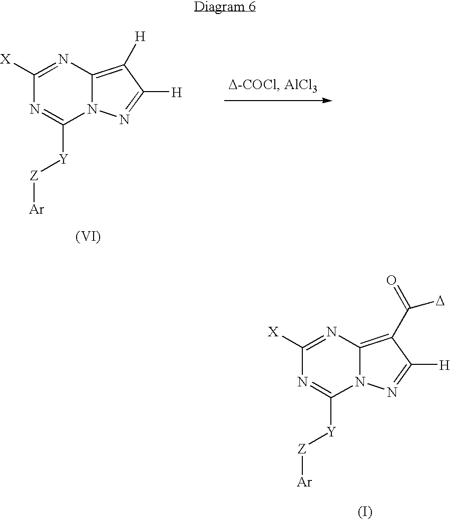

Preparation of the Compounds of General Formula (I) in which A Represents a Guanidinoaminomethylenyl of (1,3-dihydro-2-oxoindol)-3-ylidenemethyl Radical:

The compound of general formula (I) in which A represents a formyl radical is converted to the compound of general formula (I) in which A represents a guanidinoaminomethylenyl radical, Diagram 7, by reaction with aminoguanidine bicarbonate in a solvent such as ethanol and in the catalytic presence of a base such as piperidine. The compound of general formula (I) in which A represents a formyl radical is converted to the compound of general formula (I) in which A represents a (1,3-dihydro-2-oxoindol)-3-ylidenemethyl radical by the same type of reaction, the oxoindole replacing aminoguanidine bicarbonate.

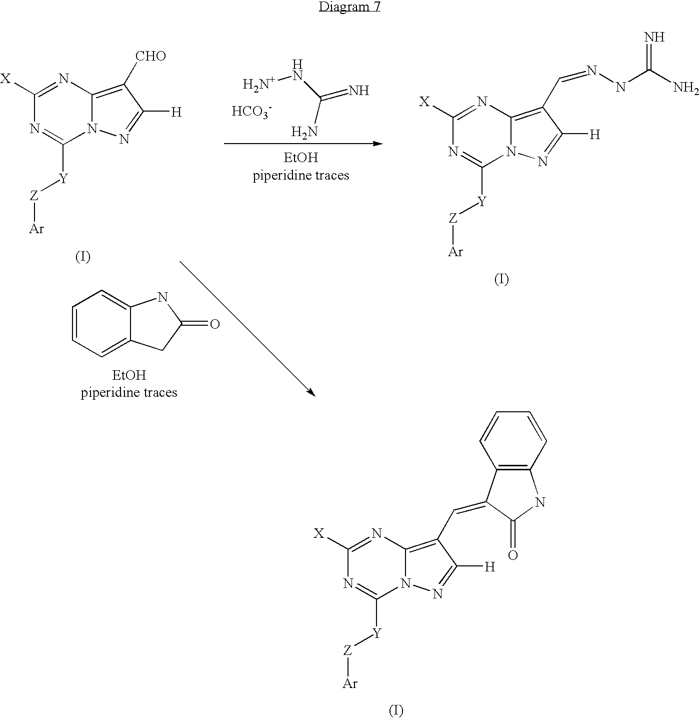

Preparation of the Compounds of General Formula (I) in which A Represents a Cyano Radical:

The compound of general formula (I) in which A represents a formyl radical is converted to the compound of general formula (I) in which A represents a cyano radical, Diagram 8, by reaction with hydroxylamine in a mixture of sodium formate and formic acid. The reaction is preferably carried out while heating.

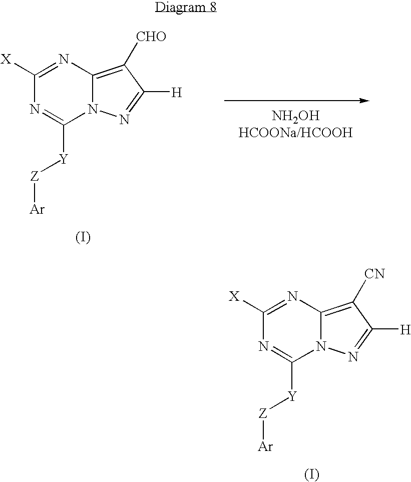

Preparation of the Compounds of General Formula (I) in which A Represents a nitro Radical:

These compounds are easily prepared from compounds of general formula (I) in which A represents a hydrogen atom by various nitration methods, for example by reacting the latter with a mixture of nitric acid and sulphuric acid or with inorganic nitrate salts in the presence of an acid such as sulphuric acid (cf. Cao et al., Synthesis (1998), 1724). Introduction of other groups (X and Y-Z-Ar) is carried out, preferably after, by using processes which are analogues to those described previously.

Unless they are defined differently, all the technical and scientific terms used here have the same meaning as that usually understood by an ordinary specialist in the field to which the invention belongs. Similarly, all publications, patent applications, patents and other references mentioned here are incorporated by way of reference.

The following examples are presented to illustrate the above procedures and should in no way be considered as limiting the scope of the invention.

### EXAMPLES

### Example 1

### 8-bromo-4-[2-(5-methyl-4-imidazolylmethylthio)-ethylamino]-2-methylthiopyrazolo[1,5-a]-1,3,5-triazine

This compound was prepared according to the method described in the American Patent U.S. Pat. No. 4,565,815. Mass spectrometry (Electrospray): 416.0.

### Example 2

### 8-bromo-4-{2-{[5-(dimethylamino)methyl-2-furannyl]-methyl}thio}ethylamino-2-methylthiopyrazolo[1,5-a]-1,3,5-triazine

This compound was prepared according to the method described in the American Patent U.S. Pat. No. 4,565,815. Mass spectrometry (Electrospray): 459.1.

### Example 3

### 8-bromo-4-(3-(1-imidazolyl-propylamino)-2-methylthio-pyrazolo[1,5-a]-1,3,5-triazine

60 μl of 1-(3-aminopropyl)imidazole is added to a solution of 8-bromo-4-chloro-2-methylthiopyrazolo[1,5-a]-1,3,5-triazine (50 mg) in a mixture of 2 ml of chloroform and 2 ml of methanol and the mixture is stirred overnight at ambient temperature. After evaporation of the solvents, the residue is divided between chloroform and water. The organic phase is then dried over MgSO₄, then, after evaporation of the solvents, the residue is subjected to preparative chromatography on silica gel using a chloroform/methanol 4/1 mixture as eluent. The appropriate fraction is isolated, extracted with a chloroform-methanol mixture and the solvents are evaporated to dryness under vacuum. A white solid is obtained. Thin layer chromatography (silica gel; chloroform/methanol in a 4/1 mixture): R_{f}=0.32. Mass spectrometry (Electrospray): 368.4; 370.1.

The compounds of Examples 4 to 11 are prepared according to a similar operating method to that of Example 3.

### Example 4

### 8-bromo-4-[(3-pyridyl)methylamino]-2-methylthio-pyrazolo[1,5-a]-1,3,5-triazine

Mass spectrometry (Electrospray): 351.0; 353.0.

### Example 5

### 8-bromo-4-(3-chloroanilino)-2-methylthio-pyrazolo[1,5-a]-1,3,5-triazine

Mass spectrometry (Electrospray): 369.9; 371.9.

### Example 6

### 8-bromo-2-methylthio-4-(pyridylmethylamino)pyrazolo[1,5-a]-1,3,5-triazine

Mass spectrometry (Electrospray): 351.0; 352.9.

### Example 7

### 8-bromo-2-methylthio-4-(2-pyridylethylamino)pyrazolo[1,5-a]-1,3,5-triazine

Mass spectrometry (Electrospray): 365.0; 366.9.

### Example 8

### 8-bromo-2-methylthio-4-(2-pyridylmethylamino)pyrazolo[1,5-a]-1,3,5-triazine

White solid. Mass spectrometry (Electrospray): 351.0; 352.9.

### Example 9

### 8-bromo-2-methylthio-4-(4-fluorophenylmethylamino)-pyrazolo[1,5-a]-1,3,5-triazine

White solid. Mass spectrometry (Electrospray): 367.9; 369.9.

### Example 10

### 8-bromo-2-methylthio-4-(3-fluorophenylmethylamino)-pyrazolo[1,5-a]-1,3,5-triazine

White solid. Mass spectrometry (Electrospray): 367.9; 369.8.

### Example 11

### 8-bromo-2-methylthio-4-[4-N-methylpiperazinyl)anilino]-pyrazolo[1,5-a]-1,3,5-triazine

White powder. Melting point: 223-224° C.

### Example 12

### 8-bromo-2-(1R-isopropyl-2-hydroxyethylamino)-4-(3-chloroanilino)-pyrazolo[1,5-a]-1,3,5-triazine

12.1) 8-bromo-4-(3-chloroanilino)-2-methylthioxo-pyrazolo[1,5-a]-1,3,5-triazine

280 mg of m-chloroperbenzoic acid is added to a solution of 8-bromo-4-(3-chloroanilino)-2-methylthio-pyrazolo[1,5-a]-1,3,5-triazine (200 mg; prepared in a similar fashion to that used for the compounds of Examples 3 to 5 starting from 8-bromo-4-chloro-2-methylthiopyrazolo[1,5-a]-1,3,5-triazine and 3-chloroaniline) in 5 ml of chloroform. The mixture is stirred overnight at ambient temperature. The reaction medium is diluted with chloroform (10 ml) and is washed with an aqueous solution of NaHSO₃ then with an aqueous solution of NaHCO₃. The organic phase is dried over MGSO₄ and the solvents are evaporated to dryness under vacuum. 200 mg of a brown solid is obtained. Mass spectrometry (Electrospray): 402.0; 404.0.

12.2) 8-bromo-2-(1R-isopropyl-2-hydroxyethylamino)-4-(3-chloroanilino)-pyrazolo[1,5-a]-1,3,5-triazine

2 ml of solution of R-Valinol in propanol (50 mg/ml) is added to a partial suspension of intermediate 12.1 (130 mg) in 5 ml of chloroform. The resultant mixture is stirred overnight at ambient temperature. After evaporation of the solvents, the residue is subjected to preparative chromatography on silica gel using a chloroform/acetone (9: 1) mixture as eluent. The appropriate fraction is isolated, extracted with a chloroform-acetone mixture and the solvents are evaporated to dryness under vacuum. A brown solid is obtained. TLC (silica gel; chloroform/acetone in 9/1 mixture): R_{f}=0.28. Mass spectrometry (Electrospray): 425.1; 427.0.

The compounds of Examples 13 to 17 are prepared according to a similar operating method to that of Example 12.

### Example 13

### 8-bromo-2-(2-aminocyclohexylamino)-4-(3-chloroanilino)-pyrazolo[1,5-a]-1,3,5-triazine

Pale yellow solid. Mass spectrometry (Electrospray): 436.1; 438.1.

### Example 14

### 8-bromo-2-(1R-isopropyl-2-hydroxyethylamino)-4-(3-oxido-pyridylmethylamino)-pyrazolo[1,5-a]-1,3,5-triazine

Pale yellow-brown liquid. Mass spectrometry=422.1.

### Example 15

### 8-bromo-2-(1R-isopropyl-2-hydroxyethylamino)-4-(3-fluorophenylmethylamino)-pyrazolo[1,5-a]-1,3,5-triazine

Mass spectrometry (Electro spray): 424.9.

### Example 16

### 8-bromo-2-(4′-hydroxyethylpiperazinyl)-4-(3-oxido-pyridylmethylamino)-pyrazolo[1,5-a]-1,3,5-triazine

Mass spectrometry (Electrospray): 451.0.

### Example 17

### 8-bromo-2-(4′-hydroxyethylpiperazinyl)-4-(3-pyridylmethylamino)-pyrazolo[1,5-a]-1,3,5-triazine

Mass spectrometry (Electrospray): 435.0.

### Example 18

### 2,4-bis-(3-pyridylmethylamino)-8-bromo-pyrazolo[1,5-a]-1,3,5-triazine

430 mg of m-chloroperbenzoic acid is added to a solution of 8-bromo-4-chloro-2-methylthio-pyrazolo[1,5-a]-1,3,5-triazine (270 mg) in 10 ml of chloroform. The mixture is stirred for one hour at ambient temperature. 4 equivalents of 3-aminomethylpyridine are added and the mixture is stirred overnight at ambient temperature. After dilution with chloroform (20 ml) and washing with water, the recovered organic phase is dried over MgSO₄. After evaporation of the solvents, the residue is subjected to preparative chromatography on silica gel using a chloroform/methanol 95/5 mixture as eluent. The appropriate fraction is isolated, extracted with a chloroform-methanol mixture and the solvents are evaporated to dryness under vacuum. A yellow solid is obtained. TLC (silica gel; chloroform/methanol in a 9/1 mixture): R_{f}=0.33. Mass spectrometry (Electrospray): 411.2; 413.2.

### Example 19

### 2,4-bis-(2-pyridylmethylamino)-8-bromo-pyrazolo[1,5-a]-1,3,5-triazine

This compound is prepared according to a similar operating method to that described for Example 17. Yellow solid. Mass spectrometry (Electrospray): 383.1; 385.1.

### Example 20

### 8-acetyl-4-(3-pyridylmethylamino)-2-methylthiopyrazolo[1,5-a]-1,3,5-triazine

213 mg of AlCl₃ then 90 μl of acetyl chloride are added successively to a solution of 2-methylthio-4-(3-pyridylmethylamino)-pyrazolo-[1,5-a]-1,3,5-triazine (110 mg) in 15 ml of dichloromethane. The mixture is taken to reflux for 4 hours. After dilution with chloroform (20 ml), the mixture is acidified with dilute HCl, then rendered basic with an aqueous solution of NaHCO₃ and the recovered organic phase is dried over MgSO₄. The solvents are eliminated by evaporation to dryness under vacuum. The residue is subjected to preparative chromatography on silica gel using a chloroform/acetone (9:1) mixture as eluent. The appropriate portions are isolated, extracted with a chloroform-methanol mixture and the solvents are eliminated by evaporation to dryness under vacuum. 65 mg of a white solid is obtained. TLC (silica gel; chloroform/acetone in a 9/1 mixture): R_{f}=0.18. Mass spectrometry (Electrospray): 315.1.

### Example 21

### 8-dimethylaminomethyl-4-(3-pyridylmethylamino)-2-methylthiopyrazolo[1,5-a]-1,3,5-triazine

A solution of 2-methylthio-4-(3-pyridylmethylamino)-pyrazolo[1,5-a]-1,3,5-triazine (50 mg) and (chloromethylene)-dimethylammonium chloride (2 equivalents) in a mixture of acetonitrile and dimethylformamide (4:1; 10 ml) is taken to reflux for 4 hours. The solvents are eliminated by evaporation to dryness under vacuum. The residue is dissolved in 20 ml of ethanol and treated with an excess of NaBH₄. After stirring for 2 hours at ambient temperature, acetic acid is added to the reaction mixture in order to decompose the excess reagent. After eliminating the solvents under vacuum, the residue is divided between CHCl₃ and water. The recovered organic phase is dried over MgSO₄. After eliminating the solvents, the residue is subjected to preparative chromatography on silica gel using a chloroform-methanol (3:1) mixture as eluent. The appropriate portions are isolated and extracted with a chloroform-methanol mixture and the solvents are eliminated by evaporation to dryness under vacuum. 19 mg of an ochre powder is obtained. TLC (silica gel; chloroform/methanol in a 3/1 mixture): R_{f}=0.19. Mass spectrometry (Electrospray): 330.1.

### Example 22

### 8-formyl-4-(3-pyridylmethylamino)-2-methylthiopyrazolo[1,5-a]-1,3,5-triazine

2-methylthio-4-(3-pyridylmethylamino)-pyrazolo[1,5-a]-1,3,5-triazine (100 mg) and (chloromethylene)-dimethylammonium chloride (4 equivalents) in an acetonitrile-dimethylformamide mixture (4:1; 50 ml) are taken to reflux for 2 hours. After evaporation of the solvents, the residue is dissolved in tetrahydrofuran (50 ml) and 25 ml of a 0.5 M aqueous solution of sodium acetate. After stirring for 4 hours at ambient temperature, the greater part of the tetrahydrofuran is eliminated under vacuum. The concentrated residue is divided between chloroform and water. The recovered organic phase is then dried over MgSO₄ and the solvents are eliminated under vacuum in order to produce 8-formyl-2-methylthio-4-(3-pyridylmethylamino)-pyrazolo[1,5-a]-1,3,5-triazine. TLC (silica gel; chloroform/methanol mixture=9/1): R_{f}=0.5. Mass spectrometry (Electrospray): 301.0.

### Example 23

### 8-morpholinomethyl-4-(3-pyridylmethylamino)-2-methylthiopyrazolo[1,5-a]-1,3,5-triazine

3 Å molecular sieves (0.5 g) and Na(OAc)₃BH (134 mg) are added to a solution of 8-formyl-4-(3-pyridylmethylamino)-2-methylthiopyrazolo[1,5-a]-1,3,5-triazine (90 mg) and morpholine (52 mg) in 40 ml of dichloroethylene containing 1% of acetic acid. The mixture obtained is stirred overnight at ambient temperature. The reaction mixture is filtered and the filtrate is diluted with chloroform (50 ml). The resultant solution is then washed with an aqueous solution of NaHCO₃ and an aqueous solution of NaCl before being dried over MgSO₄. After evaporation of the solvents, the residue is subjected to preparative chromatography on silica gel using a chloroform/methanol (9:1) mixture as eluent. The appropriate portions are isolated and extracted with a chloroform-methanol mixture and the solvents are eliminated by evaporation to dryness under vacuum. 26 mg of a whitish solid is obtained. TLC (silica gel; chloroform/methanol mixture=9/1): R_{f}=0.19. Mass spectrometry (Electrospray): 372.2.

### Example 24

### 8-[(1,3-dihydro-2-oxoindol)-3-ylidenemethyl)-2-methylthio-4-(3-pyridylmethylamino)pyrazolo[1,5-a]-1,3,5-triazine

A mixture of 8-formyl-2-methylthio-4-(3-pyridylmethylamino)-pyrazolo[1,5-a]-1,3,5-triazine (70 mg), oxoindole (64 mg) and a drop of piperidine in 50 ml of ethanol is taken to reflux for 7 hours. After returning to ambient temperature, a yellow solid is recovered by filtration and dried. TLC (silica gel; chloroform/methanol mixture=9/1: R_{f}=0.49). Mass spectrometry (Electrospray): 416.2.

### Example 25

### 8-(guanidinoaminomethylene)-2-methylthio-4-(3-pyridylmethylamino)pyrazolo [1,5-a]-1,3,5-triazine

This compound is prepared according to a similar operating method to that described for Example 24, the oxoindole being replaced by aminoguanidine bicarbonate. Brown solid. Mass spectrometry (Electrospray): 359.2.

### Example 26

### 8-bromo-2-methylthioxo-4-(3-pyridylmethylamino)pyrazolo[1,5-a]-1,3,5-triazine

This compound is prepared according to a similar operating method to that described for intermediate 12.1. Dark yellow powder. Melting point: 70-71° C.

### Example 27

### 8-bromo-2-methylthioxo-4-(3-chloroanilino)pyrazolo[1,5-a]-1,3,5-triazine

It is intermediate 12.1.

### Example 28

### 8-[(1,3-dihydro-2-oxoindol)-3-ylidenemethyl]-2-methylthio-4-[3-(1-imidazolyl)propylamino]pyrazolo[1,5-a]-1,3,5-triazine

This compound is prepared according to an operating method similar to that described for Example 24, 8-formyl-2-methylthio-4-(3-pyridylmethylamino)pyrazolo[1,5-a]-1,3,5-triazine being replaced by 8-formyl-2-methylthio-4-(3-(1-imidazolyl)propylamino) pyrazolo[1,5-a]-1,3,5-triazine. Yellow solid. Mass spectrometry (Electrospray): 433.2.

### Example 29

### 8-cyano-2-methylthio-4-(3-pyridylmethylamino)pyrazolo [1,5-a]-1,3,5-triazine

This compound is prepared by heating to reflux a mixture containing the compound of Example 22 (1 equivalent), hydroxylamine hydrochloride (2 equivalents), sodium formate (10 equivalents) and formic acid (100 equivalents) (cf. J Chem. Soc. (1965), 1564). Pale yellow solid. Mass spectrometry (Electrospray): 298.2.

### Example 30

### 8-(N-methylpiperazinomethyl)-2-methylthio-4-(3-pyridylmethylamino)pyrazolo[1,5-a]-1,3,5-triazine

This compound is prepared according to an operating method similar to that described for Example 23, morpholine being replaced by N-methylpiperazine. Brown solid. Mass spectrometry (Electrospray): 385.4; 386.4.

### Example 31: 2-methylthio-4-(3-pyridylmethylamino)pyrazolo[1,5-a]-1,3,5-triazine

3-aminomethylpyridine (3.0 g) is added to a solution of 4-chloro-2-methylthiopyrazolo[1,5-a]-1,3,5-triazine (2.0 g) in 40 ml of chloroform and 14 ml of methanol. The mixture obtained is stirred overnight at ambient temperature. After evaporation of the solvents to dryness under vacuum, the residue is divided between chloroform and the water. The organic phase is dried over MgSO₄ and the solvents are evaporated to dryness under vacuum. The residual mixture is subjected to chromatography on silica gel using a chloroform/methanol (19:1) mixture as eluent. The appropriate portions are isolated and the solvents are eliminated by evaporation to dryness under vacuum. 1.47 g of a white solid is obtained. TLC (silica gel; chloroform/methanol mixture=19/1): R_{f}=0.58. Mass spectrometry (Electrospray): 273.1.

### Example 32

### 2-methylthio-8-nitro-4-(3-pyridylmethylamino)pyrazolo[1,5-a]-1,3,5-triazine

Cupric nitrate (70 mg) is added to a suspension of 2-methylthio-4-(3-pyridylmethylamino)-pyrazolo[1,5-a]-1,3,5-triazine (50 mg; compound of Example 31) in 6 ml of acetic anhydride. The mixture is stirred at ambient temperature overnight before being divided between chloroform and a saturated aqueous solution of NaHCO₃. The organic phase is dried over MgSO₄ and the solvents are evaporated to dryness under vacuum. The residue is subjected to preparative chromatography on silica gel using a chloroform-methanol (15:1) mixture as eluent. The appropriate fraction is isolated and extracted with a chloroform methanol mixture. Once the solvents are evaporated to dryness under vacuum, the expected product is obtained in the form of a whitish solid. Thin layer chromatography (silica gel; chloroform-methanol mixture 9:1): R_{f}=0.46. Mass spectrometry (Electrospray): 318.1.

### Example 33

### 8-bromo-2-(1R-isopropyl-2-hydroxyethylamino)-4-(3-pyridylmethylamino)pyrazolo[1,5-a]-1,3,5-triazine

33.1) 8-bromo-2-methylthioxo-4-(3-pyridylmethylamino)-pyrazolo[1,5-a]-1, 3, 5-triazine

100 mg of oxone is added to a solution of 8-bromo-2-methylthio-4-(3-pyridylmethylamino)-pyrazolo[1,5-a]-1,3,5-triazine hydrochloride (100 mg) in an ethanol-water mixture (1:1; 50 ml). After 15 minutes, the mixture is diluted with water (20 ml), NaHCO₃ is added in order to render the medium basic and extraction is carried out with a chloroform-methanol (9:1) mixture. The organic phase is dried (MgSO₄) and the solvents are eliminated in order to produce the expected product in the form of a pale yellow solid (100 mg). Mass spectrometry (Electrospray): 367.2; 369.2.

33.2) 8-bromo-2-(1R-isopropyl-2-hydroxyethylamino)-4-(3-pyridylmethylamino)-pyrazolo[1,5-a]-1, 3, 5-triazine

A mixture of intermediate 33.1 (100 mg) and of R-valinol (2 eq.; 60 mg) in 3 ml of CH₃CN is taken to reflux for 3 hours. After evaporation of the solvents, the residue is taken up in a chloroform-methanol mixture (9:1; 30 ml), washed with a saturated aqueous solution of NaCl then dried over MgSO₄. The solvents are eliminated by evaporation to dryness under vacuum and the residue is subjected to preparative chromatography on silica gel using a chloroform-methanol (19:1) mixture as eluent. The appropriate fraction is isolated and extracted using a chloroform-methanol mixture. The solvents are eliminated by evaporation to dryness under vacuum. The expected product is obtained in the form of a whitish amorphous solid (50 mg). Thin layer chromatography (silica gel; chloroform-methanol mixture 9:1): R_{f}=0.32. Mass spectrometry (Electrospray): 406.2; 408.2.

Pharmacological Study of the Compounds of the Invention

Methods Used

Measurement of the State of Phosphorylation of Histone H1 by the Cyclin B1/cdc2 Complex:

The activity of the cyclin B/cyclin-dependent kinase 1 (CDK1=cdc2) complex is evaluated by the phosphorylation of a histone H1 by ATP-33 and not by ATP-32 as previously (Alessi et al., Exp. Cell Res. (1998), 245, 8-18; Baratte et al., Anticancer Res. (1992), 12, 873-880; Glab et al., FEBS Lett. (1994), 353, 207-211). The appearance of phosphorylated histone H1 in the presence of inhibitors of the CDK1 enzyme is determined by measuring the radioactivity. The cyclin B/CDK1 complex, isolated from oocytes of star-fish (Marthasterias glacialis) is purified by affinity chromatography and is then eluted with 0.2 M NaCl. Glycerol at a final concentration of 20% v/v is added to the purified enzyme before storage at −80° C. (Meijer and Kim, Methods Enzymol. (1997), 283, 113-128). The reaction is carried out in 96-well plates in a final volume of 30 μl. Each reaction contains 5 μl of histone H1 at 5 mg/ml in a final concentration (Sigma, H5505, Saint Quentin en Yvelines, France), 16 μl of buffer comprising 60 mM β-glycerophosphate (Sigma, G6251, Saint Quentin en Yvelines, France), 30 mM p-nitrophenyl phosphate (Sigma, N6260, Saint Quentin en Yvelines, France), 25 mM MOPS (Sigma M5789, Saint Quentin en Yvelines, France), 1 mM dithiothreitol (Sigma D9779, Saint Quentin en Yvelines, France), 5 mM EGTA (Sigma E8145, Saint Quentin en Yvelines, France), 0.1 mM sodium orthovanadate (Sigma S6508, Saint Quentin en Yvelines, France), 15 mM MgCl₂ (Sigma M8286, Saint Quentin en Yvelines, France) and 1 μl of the Cyclin B/CDK1 complex (final activity: 1.5 pmol of ATP incorporated in 1 minute by 1 μl of kinase). The inhibitor in an increasing concentration is added in a volume of 3 μl. The reaction starts with the addition of 5 μl of a solution of ATP containing 4 μl of ATPγ33 (370 MBq/mmol, Amersham BF1000, Les Ulis, France), 90 μl of 1 mM unlabelled ATP (Sigma A7699, Saint Quentin en Yvelines, France) diluted in 906 μl of the buffer described above.

The plates are incubated for 10 minutes at 30° C. The reaction medium is recovered on 96-well filtration plates with P81 phosphocellulose (Unifilter Polyfiltronics Whatman 7700-0512, Rungis, France) and washed with 1% TCA on a collector (Filtermate Harvester, Packard, Rungis, France). After drying the filter, Microscint® 0 scintillator (Packard, 6016311, Rungis, France) is distributed in all the wells. The radioactivity is read with a Topcount® microplate scintillation counter (Packard, Rungis, France). The results are expressed as the value of the concentration of inhibitor inhibiting 50% of the enzymatic reaction.

Measurement of the Inhibitory Activity of Glycogen Synthase Kinase-3β:

This test can be carried out as described in Leclerc et al., J Biol. Chem., September 2000.

Characterization of the Antiproliferative Activity:

By way of example, the effect of a treatment on two human cell lines Mia-Paca2 and DU145 by the compounds of Examples 1 to 33 described previously will be studied. The cell lines DU145 (human prostate cancer cells) and Mia-PaCa2 (human pancreas cancer cells) were acquired from the American Tissue Culture Collection (Rockville, Md., USA). The cells placed in 95 μl of Dulbecco's Modified Eagle's medium (Gibco-Brl, Cergy-Pontoise, France) completed with 10% foetal calf serum inactivated by heating (Gibco-Brl, Cergy-Pontoise, France), 100 units/l of penicillin and 100 μg/ml/l of streptomycin (Gibco-Brl, 10378-057, Cergy-Pontoise, France), and 2 mM of glutamine (Gibco-Brl, Cergy-Pontoise, France) were seeded on a 96-well plate on day 0. The cells were treated on day 1 for 96 hours with 5 μl of each of the compounds at concentrations increasing from 0 to 25 μM final concentration. At the end of this period, quantification of cell proliferation is evaluated by a colorimetric test based on the cleavage of the tetrazolium salt WST1 by the mitochondrial dehydrogenases in viable cells leading to the formation of formazan (Boehringer Mannheim, Meylan, France). These tests are carried out in duplicate with 8 determinations per concentration tested. For each compound to be tested, the values included in the linear part of the sigmoid were retained for a linear regression analysis and used to estimate the inhibitory concentration IC₅₀. The products are solubilized in dimethylsulphoxide (DMSO) at 10⁻² M and used in culture with 0.5% DMSO final.

Results:

The compounds of the present invention were tested according to the cyclin B/cyclin-dependent kinase 1 test described previously. All the compounds tested have shown a significant inhibition of the activity of the cyclin B/cyclin-dependent kinase 1 (CDK=cdc2).

Moreover, the compounds of the present invention were tested according to tests relating to the antiproliferative activity described previously, their activities being compared to that of Roscovitine. All the tested compounds of the present invention have shown an antiproliferative activity greater than that of Roscovitine as regards Mia-PaCa2 cells. Moreover, whilst no antiproliferative activity was observed regarding DU-145 cells with Roscovitine, numerous tested compounds of the present invention have shown an antiproliferative activity vis-à-vis these cellules as well.

1. Use of a compound of general formula (I) 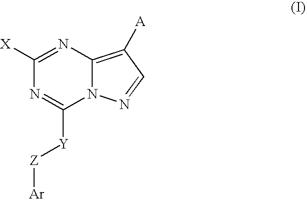
in racemic, enantiomeric form or any combination of these forms, in which
A represents a hydrogen atom, a halogen atom, a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or also an -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, complementary members being chosen independently from the group comprising —CH₂—, —NR³—, —S— and —O—, R³ representing independently each time that it occurs a hydrogen atom or an alkyl radical;
X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by a radical or radicals chosen from the alkyl, hydroxy and amino radicals, an aralkyl radical the aryl radical of which is optionally substituted by a radical or radicals chosen from a halogen atom, the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical;
Y represents NH or an oxygen atom;
Z represents a bond or an alkyl or alkylthioalkyl radical; and
Ar represents a carbocyclic aryl radical optionally substituted 1 to 3 times by radicals chosen independently from a halogen atom, the cyano radical, the nitro radical, an alkyl or alkoxy radical and an NR⁷R⁸ radical in which R⁷ and R⁸ represent independently a hydrogen atom or an alkyl radical or R⁷ and R⁸ taken together with the nitrogen atom which carries them form a heterocycle with 5 to 7 members, the complementary members being chosen independently from the group comprising —CH₂—, —NR⁹—, —S— and —O—, R⁹ representing independently each time that it occurs a hydrogen atom or an alkyl radical,
or also Ar represents a heterocyclic aryl radical containing 5 or 6 members and the heteroatom or heteroatoms of which are chosen from nitrogen, oxygen or sulphur atoms, said heteroatoms being able to be optionally oxidized and said heterocyclic aryl radical being able to be optionally substituted by a radical or radicals chosen independently from the alkyl, aminoalkyl, alkylaminoalkyl and dialkylaminoalkyl radicals;
or a pharmaceutically acceptable salt of such a compound for preparing a medicament intended to inhibit the cyclin-dependent kinases (CDK).

2. Use according to claim 1, characterized in that:
A represents a hydrogen atom, a halogen atom, a formyl, cyano, nitro, guanidinoaminomethylenyl, (1,3-dihydro-2-oxoindol)-3-ylidenemethyl, alkylcarbonyl or aralkylcarbonyl radical, or also an -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, the complementary members being chosen independently from the group comprising —CH₂—, —NR³—, —S— and —O—, R³ representing independently each time that it occurs a hydrogen atom or an alkyl radical;
X represents a hydrogen atom, an alkylthio or alkylthioxo radical, or also an NR⁴R⁵ radical in which R⁴ represents an alkyl radical, a hydroxyalkyl radical, a cycloalkyl radical optionally substituted by an amino radical or radicals, an aralkyl radical the aryl radical of which is optionally substituted by a radical or radicals chosen from a halogen atom and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical.

3. Use according to claim 1, characterized in that the compound used is chosen from the following compounds:
8-bromo-4-[2-(5-methyl-4-imidazolylmethylthio)-ethylamino]-2-methylthiopyrazolo [1,5-a]-1,3,5-triazine;
8-bromo-4-{2-{[5-(dimethylamino)methyl-2-furannyl]-methyl}thio}ethylamino-2-methylthiopyrazolo[1,5-a]-1,3,5-triazine;
8-bromo-4-(3-(1-imidazolyl-propylamino)-2-methylthio-pyrazolo[1,5-a]-1,3,5-triazine;
8-bromo-4-[(3-pyridyl)methylamino]-2-methylthio-pyrazolo[1,5-a]-1,3,5-triazine;
8-bromo-4-(3-chloroanilino)-2-methylthio-pyrazolo[1,5-a]-1,3,5-triazine;
8-bromo-2-methylthio-4-(pyridylmethylamino)pyrazolo[1,5-a]-1,3,5-triazine;
8-bromo-2-methylthio-4-(2-pyridylethylamino)pyrazolo[1,5-a]-1,3,5-triazine;
8-bromo-2-methylthio-4-(2-pyridylmethylamino)pyrazolo[1,5-a]-1,3,5-triazine;
8-bromo-2-methylthio-4-(4-fluorophenylmethylamino)-pyrazolo[1,5-a]-1,3,5-triazine;
8-bromo-2-methylthio-4-(3-fluorophenylmethylamino)-pyrazolo[1,5-a]-1,3,5-triazine;
8-bromo-2-methylthio-4-[4-N-methylpiperazinyl)anilino]-pyrazolo[1,5-a]-1,3,5-triazine;
8-bromo-2-(1R-isopropyl-2-hydroxyethylamino)-4-(3-chloroanilino)-pyrazolo[1,5-a]-1,3,5-triazine;
8-bromo-2-(2-aminocyclohexylamino)-4-(3-chloroanilino)-pyrazolo[1,5-a]-1,3,5-triazine;
8-bromo-2-(1R-isopropyl-2-hydroxyethylamino)-4-(3-oxido-pyridylmethylamino)-pyrazolo[1,5-a]-1,3,5-triazine;
8-bromo-2-(1R-isopropyl-2-hydroxyethylamino)-4-(3-fluorophenylmethylamino)-pyrazolo[1,5-a]-1,3,5-triazine;
8-bromo-2-(4′-hydroxyethylpiperazinyl)-4-(3-oxido-pyridylmethylamino)-pyrazolo[1,5-a]-1,3,5-triazine;
8-bromo-2-(4′-hydroxyethylpiperazinyl)-4-(3-pyridylmethylamino)-pyrazolo[1,5-a]-1,3,5-triazine;
2,4-bis-(3-pyridylmethylamino)-8-bromo-pyrazolo[1,5-a]-1,3,5-triazine;
2,4-bis-(2-pyridylmethylamino)-8-bromo-pyrazolo[1,5-a]-1,3,5-triazine;
8-acetyl-4-(3-pyridylmethylamino)-2-methylthiopyrazolo[1,5-a]-1,3,5-triazine;
8-dimethylaminomethyl-4-(3-pyridylmethylamino)-2-methylthiopyrazolo[1,5-a]-1,3,5-triazine;
8-formyl-4-(3-pyridylmethylamino)-2-methylthiopyrazolo[1,5-a]-1,3,5-triazine;
8-morpholinomethyl-4-(3-pyridylmethylamino)-2-methylthiopyrazolo[1,5-a]-1,3,5-triazine;
8-[(1,3-dihydro-2-oxoindol)-3-ylidenemethyl)-2-methylthio-4-(3-pyridylmethylamino)pyrazolo[1,5-a]-1,3,5-triazine;
8-(guanidinoaminomethylene)-2-methylthio-4-(3-pyridylmethylamino)pyrazolo[1,5-a]-1,3,5-triazine;
8-bromo-2-methylthioxo-4-(3-pyridylmethylamino)pyrazolo[1,5-a]-1,3,5-triazine;
8-bromo-2-methylthioxo-4-(3-chloroanilino)pyrazolo[1,5-a]-1,3,5-triazine;
8-[(1,3-dihydro-2-oxoindol)-3-ylidenemethyl)-2-methylthio-4-(3-(1-imidazolyl)propylamino)pyrazolo[1,5-a]-1,3,5-triazine;
8-cyano-2-methylthio-4-(3-pyridylmethylamino)pyrazolo[1,5-a]-1,3,5-triazine;
8-(N-methylpiperazinomethyl)-2-methylthio-4-(3-pyridylmethylamino)-pyrazolo[1,5-a]-1,3,5-triazine;
2-methylthio-4-(3-pyridylmethylamino)pyrazolo[1,5-a]-1,3,5-triazine;
2-methylthio-8-nitro-4-(3-pyridylmethylamino)pyrazolo[1,5-a]-1,3,5-triazine;
8-bromo-2-(1R-isopropyl-2-hydroxyethylamino)-4-(3-pyridylmethylamino)-pyrazolo[1,5-a]-1,3,5-triazine;
and the pharmaceutically acceptable salts of these compounds.

4. Use according to one of claims 1 to 3, characterized in that the medicament prepared is intended to treat a natural disease/disorder/phenomenon chosen from the group composed of the following natural diseases/disorders/phenomena: tumorous proliferation, the proliferation of normal cells, spontaneous alopecia, alopecia induced by exogenous products, radiation-induced alopecia, the spontaneous or induced apoptosis of normal cells, meiosis, fecundation, maturation of oocytes, viral or retroviral infections, neurodegenerative diseases, the proliferation of parasites and myopathies.

5. Use according to claim 4, characterized in that the medicament prepared is intended to treat a pathology chosen from the group comprising the following pathologies: tumorous proliferation, the proliferation of the normal cells and tauopathies.

6. As a medicament, a compound of general formula (II) 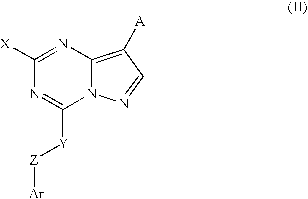
in racemic, enantiomeric form or any combination of these forms, in which
A represents a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or also an -L-NR¹R² radical in which L represents an alkylene radical and R¹ and R² are chosen independently from a hydrogen atom and an alkyl radical or. R¹ and R² taken together with the nitrogen atom which carries them form a heterocycle with 5 to 7 members, the complementary members being chosen independently from the group comprising —CH₂—, —NR³—, —S— and —O—, R³ representing independently each time that it occurs a hydrogen atom or an alkyl radical;
X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by a radical or radicals chosen from the alkyl, hydroxy and amino radicals, an aralkyl radical the aryl radical of which is optionally substituted by a radical or radicals chosen from a halogen atom, the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical;
Y represents NH or an oxygen atom;
Z represents a bond or an alkyl or alkylthioalkyl radical; and
Ar represents a carbocyclic aryl radical optionally substituted 1 to 3 times by the radicals chosen independently from a halogen atom, the cyano radical, the nitro radical, an alkyl or alkoxy radical and an NR⁷R⁸ radical in which R⁷ and R⁸ represent independently a hydrogen atom or an alkyl radical or R⁷ and R⁸ taken together with the nitrogen atom which carries them form a heterocycle with 5 to 7 members, the complementary members being chosen independently from the group comprising —CH₂—, —NR⁹—, —S— and —O—, R⁹ representing independently each time that it occurs a hydrogen atom or an alkyl radical,
or also Ar represents a heterocyclic aryl radical comprising 5 or 6 members and the heteroatom or heteroatoms of which are chosen from nitrogen, oxygen or sulphur atoms, said heteroatoms being able to be optionally oxidized and said heterocyclic aryl radical being able to be optionally substituted by a radical or radicals chosen independently from the alkyl, aminoalkyl, alkylaminoalkyl and dialkylaminoalkyl radicals;
it being understood, however, that when A does not represent a cyano, nitro or guanidinoaminomethylenyl radical then:
either Z represents an alkyl or alkylthioalkyl radical;
or X represents an NR⁴R⁵ radical in which R⁴ represents an aralkylthio, aralkylthioxo or hydroxyalkyl radical, one of the alkyl, alkylthio or alkylthioxo radicals containing 2 to 5 carbon atoms, a cycloalkyl radical optionally substituted by a radical or radicals chosen from the alkyl, hydroxy and amino radicals, an aralkyl radical the aryl radical of which is optionally substituted by a radical or radicals chosen from a halogen atom and the alkyl or alkoxy radicals, or also R⁴ represents a heteroaryl or heteroarylalkyl radical, the heteroaryl radical of the heteroaryl or heteroarylalkyl radicals being optionally substituted by an alkyl radical or radicals and R⁵ represents a hydrogen atom, or R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical; or a pharmaceutically acceptable salt of such compound.

7. Medicament characterized in that it is a compound chosen from the following compounds:
8-bromo-4-(3-(1-imidazolyl-propylamino)-2-methylthio-pyrazolo[1,5-a]-1,3,5-triazine;
8-bromo-4-[(3-pyridyl)methylamino]-2-methylthio-pyrazolo[1,5-a]-1,3,5-triazine;
8-bromo-4-(3-chloroanilino)-2-methylthio-pyrazolo[1,5-a]-1,3,5-triazine;
8-bromo-2-methylthio-4-(4-pyridylmethylamino)pyrazolo[1,5-a]-1,3,5-triazine;
8-bromo-2-methylthio-4-(2-pyridylethylamino)pyrazolo[1,5-a]-1,3,5-triazine;
8-bromo-2-methylthio-4-(2-pyridylmethylamino)pyrazolo[1,5-a]-1,3,5-triazine;
8-bromo-2-methylthio-4-(4-fluorophenylmethylamino)-pyrazolo[1,5-a]-1,3,5-triazine;
8-bromo-2-methylthio-4-(3-fluorophenylmethylamino)-pyrazolo[1,5-a]-1,3,5-triazine;
8-bromo-2-methylthio-4-[4-N-methylpiperazinyl)anilino]-pyrazolo[1,5-a]-1,3,5-triazine;
8-bromo-2-(1R-isopropyl-2-hydroxyethylamino)-4-(3-chloroanilino)-pyrazolo[1,5-a]-1,3,5-triazine;
8-bromo-2-(2-aminocyclohexylamino)-4-(3-chloroanilino)-pyrazolo[1,5-a]-1,3,5-triazine;
8-bromo-2-(1R-isopropyl-2-hydroxyethylamino)-4-(3-oxido-pyridylmethylamino)-pyrazolo[1,5-a]-1,3,5-triazine;
8-bromo-2-(1R-isopropyl-2-hydroxyethylamino)-4-(3-fluorophenylmethylamino)-pyrazolo[1,5-a]-1,3,5-triazine;
8-bromo-2-(4′-hydroxyethylpiperazinyl)-4-(3-oxido-pyridylmethylamino)-pyrazolo[1,5-a]-1,3,5-triazine;
8-bromo-2-(4′-hydroxyethylpiperazinyl)-4-(3-pyridylmethylamino)-pyrazolo[1,5-a]-1,3,5-triazine;
2,4-bis-(3-pyridylmethylamino)-8-bromo-pyrazolo[1,5-a]-1,3,5-triazine;
2,4-bis-(2-pyridylmethylamino)-8-bromo-pyrazolo[1,5-a]-1,3,5-triazine;
8-acetyl-4-(3-pyridylmethylamino)-2-methylthiopyrazolo[1,5-a]-1,3,5-triazine;
8-dimethylaminomethyl-4-(3-pyridylmethylamino)-2-methylthiopyrazolo [1,5-a]-1,3,5-triazine;
8-formyl-4-(3-pyridylmethylamino)-2-methylthiopyrazolo[1,5-a]-1,3,5-triazine;
8-morpholinomethyl-4-(3-pyridylmethylamino)-2-methylthiopyrazolo[1,5-a]-1,3,5-triazine;
8-[(1,3-dihydro-2-oxoindol)-3-ylidenemethyl)-2-methylthio-4-(3-pyridylmethylamino)pyrazolo[1,5-a]-1,3,5-triazine;
8-(guanidinoaminomethylene)-2-methylthio-4-(3-pyridylmethylamino)pyrazolo[1,5-a]-1,3,5-triazine;
8-bromo-2-methylthioxo-4-(3-pyridylmethylamino)pyrazolo[1,5-a]-1,3,5-triazine;
8-bromo-2-methylthioxo-4-(3-chloroanilino)pyrazolo[1,5-a]-1,3,5-triazine;
8-[(1,3-dihydro-2-oxoindol)-3-ylidenemethyl)-2-methylthio-4-(3-(1-imidazolyl)propylamino)pyrazolo[1,5-a]-1,3,5-triazine;
8-cyano-2-methylthio-4-(3-pyridylmethylamino)pyrazolo[1,5-a]-1,3,5-triazine;
8-(N-methylpiperazinomethyl)-2-methylthio-4-(3-pyridylmethylamino)-pyrazolo[1,5-a]-1,3,5-triazine;
2-methylthio-4-(3-pyridylmethylamino)pyrazolo[1,5-a]-1,3,5-triazine;
2-methylthio-8-nitro-4-(3-pyridylmethylamino)pyrazolo [1,5-a]-1,3,5-triazine;
8-bromo-2-(1R-isopropyl-2-hydroxyethylamino)-4-(3-pyridylmethylamino)-pyrazolo[1,5-a]-1,3,5-triazine;
and the pharmaceutically acceptable salts of these compounds.

8. As a new industrial product, a compound of general formula (II) as defined in claim 6 or a salt of such compound.

9. Product characterized in that it is a compound chosen from the following compounds:
8-bromo-4-(3-(1-imidazolyl-propylamino)-2-methylthio-pyrazolo[1,5-a]-1,3,5-triazine;
8-bromo-4-[(3-pyridyl)methylamino]-2-methylthio-pyrazolo[1,5-a]-1,3,5-triazine;
8-bromo-4-(3-chloroanilino)-2-methylthio-pyrazolo [1,5-a]-1,3,5-triazine;
8-bromo-2-methylthio-4-(4-pyridylmethylamino)pyrazolo[1,5-a]-1,3,5-triazine;
8-bromo-2-methylthio-4-(2-pyridylethylamino)pyrazolo[1,5-a]-1,3,5-triazine;
8-bromo-2-methylthio-4-(2-pyridylmethylamino)pyrazolo[1,5-a]-1,3,5-triazine;
8-bromo-2-methylthio-4-(4-fluorophenylmethylamino)-pyrazolo[1,5-a]-1,3,5-triazine;
8-bromo-2-methylthio-4-(3-fluorophenylmethylamino)-pyrazolo [1,5-a]-1,3,5-triazine;
8-bromo-2-methylthio-4-[4-N-methylpiperazinyl)anilino]-pyrazolo[1,5-a]-1,3,5-triazine;
8-bromo-2-(1R-isopropyl-2-hydroxyethylamino)-4-(3-chloroanilino)-pyrazolo[1,5-a]-1,3,5-triazine;
8-bromo-2-(2-aminocyclohexylamino)-4-(3-chloroanilino)-pyrazolo[1,5-a]-1,3,5-triazine;
8-bromo-2-(1R-isopropyl-2-hydroxyethylamino)-4-(3-oxido-pyridylmethylamino)-pyrazolo[1,5-a]-1,3,5-triazine;
8-bromo-2-(1R-isopropyl-2-hydroxyethylamino)-4-(3-fluorophenylmethylamino)-pyrazolo[1,5-a]-1,3,5-triazine;
8-bromo-2-(4′-hydroxyethylpiperazinyl)-4-(3-oxido-pyridylmethylamino)-pyrazolo[1,5-a]-1,3,5-triazine;
8-bromo-2-(4′-hydroxyethylpiperazinyl)-4-(3-pyridylmethylamino)-pyrazolo[1,5-a]-1,3,5-triazine;
2,4-bis-(3-pyridylmethylamino)-8-bromo-pyrazolo [1,5-a]-1,3,5-triazine;
2,4-bis-(2-pyridylmethylamino)-8-bromo-pyrazolo [1,5-a]-1,3,5-triazine;
8-acetyl-4-(3-pyridylmethylamino)-2-methylthiopyrazolo [1,5-a]-1,3,5-triazine;
8-dimethylaminomethyl-4-(3-pyridylmethylamino)-2-methylthiopyrazolo[1,5-a]-1,3,5-triazine;
8-formyl-4-(3-pyridylmethylamino)-2-methylthiopyrazolo[1,5-a]-1,3,5-triazine;
8-morpholinomethyl-4-(3-pyridylmethylamino)-2-methylthiopyrazolo[1,5-a]-1,3,5-triazine;
8-[(1,3-dihydro-2-oxoindol)-3-ylidenemethyl)-2-methylthio-4-(3-pyridyhnethylamino)pyrazolo[1,5-a]-1,3,5-triazine;
8-(guanidinoaminomethylene)-2-methylthio-4-(3-pyridylmethylamino)pyrazolo[1,5-a]-1,3,5-triazine;
8-bromo-2-methylthioxo-4-(3-pyridylmethylamino)pyrazolo [1,5-a]-1,3,5-triazine;
8-bromo-2-methylthioxo-4-(3-chloroanilino)pyrazolo[1,5-a]-1,3,5-triazine;
8-[(1,3-dihydro-2-oxoindol)-3-ylidenemethyl)-2-methylthio-4-(3-(1-imidazolyl)propylamino)pyrazolo[1,5-a]-1,3,5-triazine;
8-cyano-2-methylthio-4-(3-pyridylmethylamino)pyrazolo[1,5-a]-1,3,5-triazine;
8-(N-methylpiperazinomethyl)-2-methylthio-4-(3-pyridylmethylamino)-pyrazolo[1,5-a]-1,3,5-triazine;
2-methylthio-4-(3-pyridylmethylamino)pyrazolo[1,5-a]-1,3,5-triazine;
2-methylthio-8-nitro-4-(3-pyridylmethylamino)pyrazolo[1,5-a]-1,3,5-triazine;
8-bromo-2-(1R-isopropyl-2-hydroxyethylamino)-4-(3-pyridylmethylamino)-pyrazolo[1,5-a]-1,3,5-triazine;
and the salts of these compounds.

10. Pharmaceutical composition comprising, as active ingredient, a compound of general formula (II) as defined in claim 6, or a pharmaceutically acceptable salt of one of the latter.
